(19) United States
(12) Patent Application Publication
(10) Pub. No.: US 2014/0187596 A1
(43) Pub. Date: Jul. 3, 2014

(54) FATTY ACID AMIDE HYDROLASE INHIHIBITORS FOR TREATING PAIN

(71) Applicant: ALLERGAN, INC., Irvine, CA (US)

(72) Inventors: David F. Woodward, Lake Forest, CA (US); Jose L. Martos, Basildon Essex (GB); William R. Carling, Bishop's Stortford (GB); Andrew D. Jones, Saffron Walden (GB); Jenny W. Wang, IRVINE, CA (US)

(73) Assignee: ALLERGAN, INC., IRVINE, CA (US)

(21) Appl. No.: 14/119,803

(22) PCT Filed: May 23, 2012
(86) PCT No.: PCT/US2012/039185

§ 371 Date: Feb. 19, 2014

Related U.S. Application Data
(60) Provisional application No. 61/489,841, filed on May 25, 2011.

(51) Int. Cl. C07D 493/08

(52) U.S. Cl. CPC C07D 493/08
USPC 514/374; 548/236

(57) ABSTRACT

The present invention provides a method of treating a patient suffering from pain or other FAAH mediated conditions by administering a fatty acid amide inhibiting amount of a compound represented by the formula: wherein R₁ is H; R₂ is a radical selected from the group consisting of H, hydrocarbyl and substituted hydrocarbyl; R₃ is a radical selected from the group consisting of H, hydrocarbyl and substituted hydrocarbyl; X is CHCH, (CH₂)ₙ or O(CH₂)ₙ, wherein n is 0 or an integer of from 1 to 4; and W is O, S, or NR⁶, wherein R⁶ is selected from the group consisting of H and alkyl.

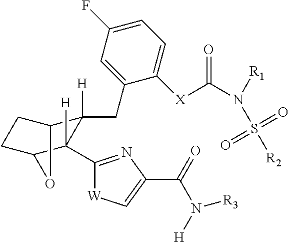

### CROSS-REFERENCE TO RELATED APPLICATION

This application is based, and claims priority under 35 U.S.C. §120 to U.S. Provisional Patent Application No. 61/489,841 filed on May 25, 2011, and which is incorporated herein by reference.

### BACKGROUND OF THE INVENTION

1. Field of the Invention

This invention relates to a method for treating pain and other diseases and conditions of the central nervous system (CNS) and peripheral nervous system (PNS) by inhibiting the action of fatty acid amide hydrolase in the body of a patient in need of treatment therefore to thereby modulate the breakdown of naturally occurring endocannabinoids, such as anandamide. In addition, blockade of prostanoid receptors provides additional benefit.

2. Background of the Art

Fatty acid amide hydrolase (FAAH) is an enzyme that modulates central nervous system (CNS) functions such as pain perception, cognition, feeding, sleep and locomotion by breaking down certain fatty signaling molecules that reside in the lipid membranes of CNS cells

The structure of this enzyme was described in the journal, Science, by researchers from the Scripps Institute. The Scripps researchers reported that FAAH modulates the action of these fatty signaling molecules through an unusual mechanism whereby it “scoops” such molecules out of the cell membranes and “chews” them up. The researchers surmised that the deep pocket with well-defined cavities provided the guidance to take the currently available tight binding inhibitors and improve on their specificity and pharmacokinetic properties. The researchers also surmised that a specific inhibitor of FAAH could, in principle, provide pain relief without any side effects.

There is an ongoing search for compounds that not only ease pain, but do so as fast, effectively and long-lasting as possible, and without any unwanted side effects; however every analgesic, from opiates to hypnotism to electroshocks to balms, has side effects.

Delta-9-tetrahydrocannabinol (THC), the active ingredient in marijuana, works as an analgesic by mimicking the action of natural cannabinoids that the body produces in signaling cascades in response to a peripheral pain stimulus. THC binds to “CB-1” receptors on cells on the rostral ventromedial medulla, a pain-modulating center of the brain, decreasing sensitivity to pain. However, the receptors that THC binds to are also widely expressed in other parts of the brain, such as the memory and information-processing centers of the hippocampus. Binding to nerve cells of the hippocampus and other cells elsewhere in the body, THC creates a range of side effects as it activates CB-1 mediated signaling, including distorted perception, difficulty in problem-solving, loss of coordination, and increased heart rate and blood pressure, anxiety and panic attacks. Thus, the challenge thus posed by THC and other cannabinoids is to find a way to use them to produce effective, long-lasting relief from pain without the deleterious side effects.

It has been suggested that the solution is to increase the efficacy of the natural, endogenous cannabinoids (“endocannabinoids”) the body produces to modulate pain sensations. The amplitude and duration of the activity of such endocannabinoids are regulated by how fast they are broken down. In particular, the body releases an endogenous cannabinoid called anandamide. When the body senses pain, anandamide binds to CB-1 and nullifies pain by blocking the signaling. However, this effect is weak and short-lived as FAAH quickly metabolizes anandamide, as the compound has a half-life of only a few minutes in vivo.

In some ways, THC is superior to anandamide as a pain reliever because it is not as readily metabolized by FAAH. But, since THC goes on to suppress cannabinoid receptor activity all over the body and it is a controlled substance, THC is an unattractive target for developing therapeutics, as compared to FAAH.

FAAH is much more attractive target for pain therapy because by inhibiting FAAH, you would increase the longevity of anandamide molecules, preventing their breakdown and allowing them to continue providing some natural pain relief. Thus, the design of specific inhibitors that would control the action of FAAH when the body is sensing pain and releasing anandamide is very desirable.

### SUMMARY OF THE INVENTION

The present invention provides a method for inhibiting the activity of fatty acid amide hydrolase (FAAH) and multiple prostanoid receptors in a human to thereby modulate central nervous system (CNS) functions such as pain perception, cognition, feeding, sleep, and locomotive activity. The method of the present invention functions to break down certain fatty signaling molecules that reside in the lipid membranes of CNS cells by treating a patient in need of said treatment with an effective amount of a compound represented by the formula:

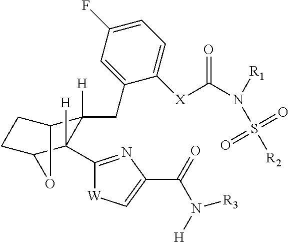

wherein R₁ is H;

R₂ is a radical selected from the group consisting of H, hydrocarbyl and substituted hydrocarbyl, e.g. H, alkyl, haloalkyl and aryl;

R₃ is a radical selected from the group consisting of H, hydrocarbyl and substituted hydrocarbyl, e.g. H, alkyl, alkenyl and aryl, i.e. n-alkyl or cycloalkyl-n-alkyl;

X is CHCH, (CH₂)ₙ or O(CH₂)ₙ, wherein n is 0 or an integer of from 1 to 4; and,

W is O, S, or NR⁶, wherein R⁶ is selected from the group consisting of H and alkyl.

Some embodiments of the invention include:

1. A method of treating a patient suffering from pain by administering a fatty acid amide inhibiting amount of a compound represented by the formula:

wherein R1 is H;

R2 is a radical selected from the group consisting of H, hydrocarbyl and substituted hydrocarbyl;

R3 is a radical selected from the group consisting of H, hydrocarbyl and substituted hydrocarbyl;

X is CHCH, (CH2)n or O(CH2) n, wherein n is 0 or an integer of from 1 to 4; and

W is O, S, or NR6, wherein R6 is selected from the group consisting of H and alkyl.

2. The method of embodiment 1 wherein W is O.

3. The method of embodiments 1 and 2 wherein R2 is a radical selected from the group consisting of H, alkyl, haloalkyl and aryl.

4. The method of embodiments 1 and 2 wherein R2 is selected from the group consisting of ethyl, methyl, 2-methylethyl, phenyl, trifluoromethyl and 2, 2, 2 trifluoroethyl.

5. The method of embodiment 1 wherein R3 is selected from the group consisting of H, alkyl, alkenyl and aryl.

6. The method of embodiments 1, 2 and 4 wherein R3 is selected from the group consisting of n-alkyl and cycloalkyl-n-alkyl

7. The method of embodiments 1 and 5 wherein, R3 is (CH2)nCH2R5, wherein n is an integer of from 4 to 9 and R5 is H or cycloalkyl.

8. The method of embodiment 6 wherein R3 is selected from the group consisting of cyclohexyl-n-alkyl radicals.

9. The method of embodiments 1, 2 and 7 wherein R3 is cyclohexyl-n-butyl.

10. The method of embodiments 1 and 2 wherein X is ethyl or ethenyl.

11. The method of embodiments 1 and 2 wherein said compound is selected from the group consisting of

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(ethylsulfonyl)acrylic amide,

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(methylsulfonyl)acrylic amide,

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2,2,2-trifluoroethanesulfonyl)acrylic amide,

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2-methylethanesulfonyl)acrylic amide,

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(benzenesulfonyl)acrylic amide,

(E)-3R-[2R-[[3-[4-[[(octylamino]carbonyl]-2-oxazolyl]-7-oxabicyclo[2.2.1]hept-2-yl]methyl]-4-fluoro-phenyl]-N-(ethylsulfonyl)acrylic amide, and

2-{3-[5-Fluoro-2-(3-oxo-3-trifluoromethanesulfonylamino-propyl)-benzyl]-7-oxa-bicyclo[2.2.1]hept-2-yl}-oxazole-4-carboxylic acid nonylamide.

12. A method of treating a patient having a condition mediated by FAAH which comprises administering a fatty acid amide inhibiting amount of a compound represented by the formula:

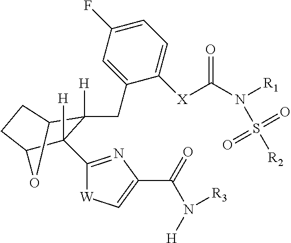

wherein R1 is H;

R2 is a radical selected from the group consisting of H, hydrocarbyl and substituted hydrocarbyl;

R3 is a radical selected from the group consisting of H, hydrocarbyl and substituted hydrocarbyl;

X is CHCH, (CH2)n or O(CH2) n, wherein n is 0 or an integer of from 1 to 4; and

W is O, S, or NR6, wherein R6 is selected from the group consisting of H and alkyl.

13. A method of treating a patient having a condition mediated by FAAH and at least one PG receptor which comprises administering a fatty acid amide inhibiting amount of a compound represented by the formula:

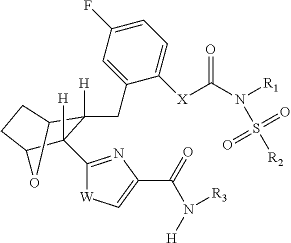

wherein R1 is H;

R2 is a radical selected from the group consisting of H, hydrocarbyl and substituted hydrocarbyl;

R3 is a radical selected from the group consisting of H, hydrocarbyl and substituted hydrocarbyl;

X is CHCH, (CH2)n or O(CH2) n, wherein n is 0 or an integer of from 1 to 4; and

W is O, S, or NR6, wherein R6 is selected from the group consisting of H and alkyl.

14. The method of embodiments 12 and 13 wherein said condition is a pain-related condition.

15. A method of treating a patient suffering from pain by administering a fatty acid amide inhibiting amount of a compound, that is a 3-[4-[[(hydrocarbyl or substituted hydrocarbyl)amino]carbonyl]-2-oxazolyl]-7-oxabicyclo[2.2.1]hept-2-yl]methyl]-substitutedphenyl]-N-(ethylsulfonyl)acrylic amide.

16. The method of embodiment 15 wherein said compound is a 3-[4-[[(alkyl)amino]carbonyl]-2-oxazolyl]-7-oxabicyclo[2.2.1]hept-2-yl]methyl]-4-substituted-phenyl]-N-(ethylsulfonyl)acrylic amide.

17. The method of embodiment 15 wherein said compound is a 3-[4-[[(alkyl)amino]carbonyl]-2-oxazolyl]-7-oxabicyclo[2.2.1]hept-2-yl]methyl]-4-halo-phenyl]-N-(ethylsulfonyl)acrylic amide.

18. A compound represented by the formula:

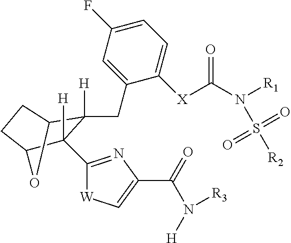

wherein R1 is H;

R2 is a radical selected from the group consisting of H, hydrocarbyl and substituted hydrocarbyl;

R3 is a radical selected from the group consisting of H, hydrocarbyl and substituted hydrocarbyl;

X is CHCH, (CH2)n or O(CH2) n, wherein n is 0 or an integer of from 1 to 4; and

W is O, S, or NR6, wherein R6 is selected from the group consisting of H and alkyl.

19. The embodiment of claim 18 wherein W is O.

20. The compound of embodiments 18 and 19 wherein R2 is a radical selected from the group consisting of H, alkyl, haloalkyl and aryl.

21. The compound of embodiments 18 and 19 wherein R2 is selected from the group consisting of ethyl, methyl, 2-methylethyl, phenyl, trifluoromethyl and 2, 2, 2 trifluoroethyl.

22. The compound of embodiments 18 and 19 wherein R3 is selected from the group consisting of H, alkyl, alkenyl and aryl.

23. The compound of embodiment 21 wherein R3 is selected from the group consisting of n-alkyl and cycloalkyl-n-alkyl

24. The compound of embodiment 22 wherein R3 is (CH2)nCH2R5, wherein n is an integer of from 4 to 9 and R5 is H or cycloalkyl.

25. The compound of embodiment 23 wherein R3 is selected from the group consisting of cyclohexyl-n-alkyl radicals.

26. The compound of embodiments 18 and 19 wherein R3 is cyclohexyl-n-butyl.

27. The compound of embodiment 18 wherein X is ethyl or ethenyl.

28. The compound of embodiments 18 and 19 wherein said compound is selected from the group consisting of:

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(ethylsulfonyl)acrylic amide;

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(methylsulfonyl)acrylic amide;

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2,2,2-trifluoroethanesulfonyl)acrylic amide;

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2-methylethanesulfonyl)acrylic amide;

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(benzenesulfonyl)acrylic amide;

(E)-3R-[2R-[[3-[4-[[(octylamino]carbonyl]-2-oxazolyl]-7-oxabicyclo[2.2.1]hept-2-yl]methyl]-4-fluoro-phenyl]-N-(ethylsulfonyl)acrylic amide; and,

2-{3-[5-Fluoro-2-(3-oxo-3-trifluoromethanesulfonylamino-propyl)-benzyl]-7-oxa-bicyclo[2.2.1]hept-2-yl}-oxazole-4-carboxylic acid nonylamide.

### DETAILED DESCRIPTION OF THE INVENTION

Definitions:

Unless otherwise stated the following terms used in the specification and claims have the meanings discussed below:

“Hydrocarbyl” refers to a hydrocarbon radical having only carbon and hydrogen atoms. Preferably, the hydrocarbyl radical has from 1 to 20 carbon atoms, more preferably from 1 to 12 carbon atoms and most preferably from 1 to 7 carbon atoms.

“Substituted hydrocarbyl” refers to a hydrocarbyl radical wherein one or more, but not all, of the hydrogen and/or the carbon atoms are replaced by a halogen, nitrogen, oxygen, sulfur or phosphorus atom or a radical including a halo, nitrogen, oxygen, sulfur or phosphorus atom, e.g. fluoro, chloro, cyano, nitro, dialkylamino, hydroxyl, phosphate, thiol, etc.

“Alkyl” refers to a straight-chain, branched or cyclic saturated aliphatic hydrocarbon. Preferably, the alkyl group has 1 to 20 carbons, more preferably from 1 to 12 carbons and most preferably 1 to 10 carbons. Typical alkyl groups include methyl, ethyl, n-propyl, isopropyl, butyl, isobutyl, tertiary butyl, pentyl, hexyl and the like as well as cycloalkyl-n-alkyl groups such as cyclohexyl-n-butyl. The alkyl group may be optionally substituted with one or more substituents selected from the group consisting of hydroxyl, cyano, alkoxy, ═O, ═S, NO₂, halo, dimethyl amino, and SH.

“Alkenyl” refers to a straight-chain, branched or cyclic unsaturated aliphatic hydrocarbon having one or more carbon-carbon double bonds. Preferably, the alkenyl group has 2 to 20 carbons, more preferably from 2 to 12 carbons and most preferably 2 to 10 carbons. Preferably, the alkenyl group has one carbon-carbon double bond. Typical alkylenyl groups include ethylenyl, propylenyl, butylenyl, pentylenyl, hexylenyl and the like as well as cycloalkylenyl groups such as cyclohexenyl-n-butyl. The alkylenyl group may be optionally substituted with one or more substituents selected from the group consisting of hydroxyl, cyano, alkoxy, ═O, ═S, NO₂, halo, dimethyl amino, and SH.

“Cycloalkyl” refers to a cyclic saturated aliphatic hydrocarbon group. Preferably, the cycloalkyl group has 3 to 12 carbons. More preferably, it has from 4 to 7 carbons, most preferably 5 or 6 carbons.

“Aryl” refers to an aromatic group which has at least one ring having a conjugated pi electron system and includes carbocyclic aryl, heterocyclic aryl and biaryl groups. The aryl group may be optionally substituted with one or more substituents selected from the group consisting of alkyl, hydroxyl, halo, COOR⁶, NO₂, CF₃, N(R⁶)₂, CON(R⁶)₂, SR⁶, sulfoxy, sulfone, CN and OR⁶, wherein R⁶ is alkyl.

“Carbocyclic aryl” refers to an aryl group wherein the ring atoms are carbon.

“Heteroaryl” or “heterocyclic aryl” refers to a monocyclic or fused ring (i.e., rings which share an adjacent pair of atoms) group of 5 to 12 ring atoms containing one, two, three or four ring heteroatoms selected from N, O, or S, the remaining ring atoms being C, and, in addition, having a completely conjugated pi-electron system. Examples, without limitation, of heteroaryl groups are pyrrole, furan, thiophene, imidazole, oxazole, thiazole, pyrazole, pyridine, pyrimidine, quinoline, isoquinoline, purine, tetrazole, triazine, and carbazole. The heteroaryl group may be substituted or unsubstituted.

“Hydroxyl” refers to an —OH group.

“Alkoxy” refers to an —O-(alkyl) an —O-(cycloalkyl) or an —O-alkyl-O— group. Representative examples include, but are not limited to, e.g., methoxy, ethoxy, propoxy, butoxy, dioxol, cyclopropyloxy, cyclobutyloxy, cyclopentyloxy, cyclohexyloxy, and the like.

“Acyl” refers to a —C(O)— group.

“Halo” refers to fluorine, chlorine, bromine or iodine, preferably fluorine or chlorine.

“Dialkylamino” means a radical —NRR where each R is independently an alkyl or cycloalkyl group as defined above, e.g., dimethylamino, diethylamino, (1-methylethyl)-ethylamino, cyclohexylmethylamino, cyclopentylmethylamino, and the like.

“Optional” or “optionally” means that the subsequently described event or circumstance may but need not occur, and that the description includes instances where the event or circumstance occurs and instances in which it does not. For example, “heterocycle group optionally substituted with an alkyl group” means that the alkyl may but need not be present, and the description includes situations where the heterocycle group is substituted with an alkyl group and situations where the heterocyclo group is not substituted with the alkyl group.

As set forth above, the present invention provides a method of treating pain, defects in cognition and locomotive activity, problems with feeding, sleeping, etc. by treating a patient in need of said treatment with an effective amount of a compound represented by the formula, above.

Preferably, said compound is represented by the formula, below:

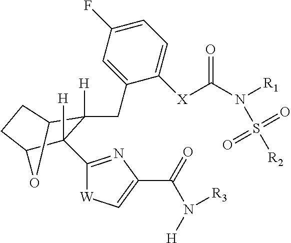

wherein R₁, R₂, R₃, R₆, X and W are as defined above.

Preferably, R2 is alkyl, including cycloalkyl, fluoroalkyl or carbocyclic aryl, including phenyl.

More preferably, R₂ is selected from the group consisting of ethyl, methyl, 2-methylethyl, phenyl, trifluoromethyl and 2, 2, 2 trifluoroethyl.

Preferably, R₃ is an alkyl radical, including cycloalkyl-n-alkyl radicals.

More preferably, R₃ is (CH₂)ₙCH₂R₅, wherein n is an integer of from 4 to 9 and R₅ is H or cycloalkyl.

Even more preferably, R₃ is selected from the group consisting of cyclohexyl-n-alkyl radicals.

Most preferably, R₃ is cyclohexyl-n-butyl.

Preferably, X is ethyl or ethenyl, more preferably ethenyl.

Preferably W is O.

The most preferred compounds for use in the method of the present invention are selected from the group consisting of:

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(ethylsulfonyl)acrylic amide;

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(methylsulfonyl)acrylic amide;

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2,2,2-trifluoroethanesulfonyl)acrylic amide;

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2-methylethanesulfonyl)acrylic amide;

(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(benzenesulfonyl)acrylic amide;

(E)-3R-[2R-[[3-[4-[[(octylamino]carbonyl]-2-oxazolyl]-7-oxabicyclo[2.2.1]hept-2-yl]methyl]-4-fluoro-phenyl]-N-(ethylsulfonyl)acrylic amide; and,

2-{3-[5-Fluoro-2-(3-oxo-3-trifluoromethanesulfonylamino-propyl)-benzyl]-7-oxa-bicyclo[2.2.1]hept-2-yl}-oxazole-4-carboxylic acid nonylamide.

The invention further relates to pharmaceutical compositions containing the above compounds in combination with a pharmaceutically-acceptable excipient and to their use in medicine, in particular their use in the treatment of conditions mediated by the action of the FAAH enzyme and, additionally, ligands for the DP₁, FP, EP₁, EP₃ and EP₄ prostaglandin (PG) receptors. The compounds of this invention are also useful for treating conditions mediated by the action of ligands for the thromboxane (TP) receptor.

As shown in the following tables, the compounds of this invention are, also, pan antagonists of the PG receptors, having particular activity at the FP, DP, EP₁, EP₃, EP₄ and TP receptors, but are much less active at the EP₂ and IP receptors. Thus, these compounds have a biological selectivity profile making them useful in treating diseases and conditions which are mediated by the FP, DP, EP₁, EP₃, EP₄ and TP receptors, without the potential side effects and biological limitations associated with IP and/or EP₂ receptor blockade. Thus, the compounds of this invention compound may be also administered to treat DP₁, FP, EP₁, EP₃, TP and/or EP₄ receptor mediated diseases or conditions, as well as diseases mediated by FAAH.

For example, said condition or disease may be related to inflammation, or said DP₁, FP, EP₁, EP₃, TP and/or EP₄ receptor mediated condition, or the disease or condition may be selected from the group consisting of allergic conditions, asthma, allergic asthma, apnea, allergic conjunctivitis, allergic rhinitis, atopic dermatitis, uveitis and related disorders, atherosclerosis, blood coagulation disorders, bone disorders, cancer, cellular neoplastic transformations, chronic obstructive pulmonary diseases and other forms of lung inflammation, pneumonia, congestive heart failure, diabetic retinopathy, diseases or conditions requiring a treatment of anti-coagulation, diseases requiring control of bone formation and resorption, fertility disorders, pre-term labor, endometriosis, glaucoma, hyperpyrexia, immune and autoimmune diseases, inflammatory conditions, metastic tumor growth, migraine, mucus secretion disorders, nasal congestion, nasal inflammation, occlusive vascular diseases, ocular hypertension, ocular hypotension, osteoporosis, rheumatoid arthritis, pain, perennial rhinitis, pulmonary congestion, pulmonary hypotension, Raynaud's disease, rejection in organ transplant and by-pass surgery, respiratory conditions, hirsutism, rhinorrhea, shock, sleep disorders, and sleep-wake cycle disorders, over active bladder disorders.

Said compounds may be administered as a surgical adjunct in ophthalmology for cataract removal and artificial lens insertion, ocular implant procedures, photorefractive radial keratotomy and other ophthalmogical laser procedures or as a surgical adjunct in a procedure involving skin incisions, relief of pain and inflammation and scar formation/keloids post-surgery, for treating sports injuries and general aches and pains in muscles and joints. Said DP₁, FP, EP₁, EP3, TP, and/or EP₄ receptor mediated condition or disease may be an EP₁ and/or EP₄ receptor mediated condition or disease.

Said DP₁, FP, EP₁, EP₃, TP and/or EP₄ receptor mediated condition or disease may be an allergic condition, e.g. an dermatological allergy, or an ocular allergy, or a respiratory allergy, e.g. nasal congestion, rhinitis, and asthma. Said condition or disease may be a bleeding disorder, or a sleep disorder, or mastocytosis.

Said DP₁, FP, EP₁, EP₃, TP and/or EP₄ receptor mediated condition or disease may be associated with elevated body temperature, or ocular hypertension and glaucoma, or ocular hypotension. In particular, said DP₁, FP, EP₁, EP₃, TP and/or EP₄ receptor mediated condition or disease may be related to pain. Therefore, the compounds utilized in the method of this invention may treat pain by two or more mechanisms, simultaneously, i.e. by inhibiting FAAH and antagonizing the appropriate PG receptor, simultaneously.

Said pain-related condition or disease may be selected from the group consisting of arthritis, migraine, and headache.

Said pain-related condition or disease may be associated with the gastrointestinal tract, wherein said condition or disease may be peptic ulcer, heartburn, reflux esophagitis, erosive esophagitis, non-ulcer dyspepsia, infection by Helicobacter pylori, alrynitis, and irritable bowel syndrome. Said pain-related condition or disease may be selected from the group consisting of hyperalgesia and allodynia, or said condition or disease may be related to mucus secretion, wherein said mucus secretion is gastrointestinal, or occurs in the nose, sinuses, throat, or lungs. Said pain-related condition or disease is related to abdominal cramping, e.g. said condition or disease may be irritable bowel syndrome. Said condition may relate to surgical procedures to treat pain, inflammation and other unwanted sequelae wherein said surgical procedure includes incision, laser surgery or implantation. Finally, said condition may be related to pain and inflammation and post-surgical scar and keloid formation

The following examples are intended to further illustrate this invention and describe the best mode of practicing the method of the invention.

### EXAMPLE 1

### (E)-3-(2R-{3R-[4-(4-Cyclohexyl-butylcarbamoyl)-oxazol-2-yl]-7-oxa-bicyclo[2.2.1]hept-2-ylmethyl}-4-fluoro-phenyl)-acrylic acid

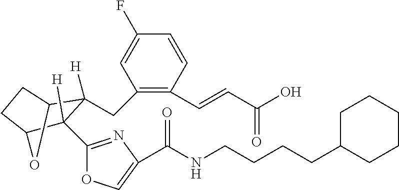 [SMILES: O=C(O)/C=C/c1ccc(F)cc1CC1C2CCC(O2)C1c1nc(C(=O)NCCCCC2CCCCC2)co1]

To a solution 2-bromo-4-fluorobenzaldehyde (15.2 g, 74.9 mmol) in toluene (80 ml) was added (1R,2R)-(−)-pseudoephedrine (13.6 g, 82 mmol) and the resulting mixture was refluxed removing water using a Dean-Stark trap for 16 h. The reaction was halted and cooled down to room temperature. The solution was washed with citric acid solution (1M, 100 ml), saturated sodium bicarbonate solution (50 ml), brine (50 ml) and dried (MgSO₄). Then, it was filtered and the solvent was evaporated under vacuum to give the title compound as a yellow oil. (26.2 g, yield=97%).

¹H-NMR (CDCl₃, 300 MHz) δ 7.78 (dd, 1H, J=5.7, 8.6 Hz, ArH), 7.36 (m, 6H, ArH), 7.11 (m, 1H, ArH), 5.47 (s, 1H, —N—CH—O—), 4.71 (d, 1H, J=8.64 Hz, —CH-Ph), 2.60 (m, 1H, —CH—CH₃), 2.27 (s, 3H, —CHCH₃). ¹⁹F-NMR (CDCl₃, 300 MHz) δ −111.6

### Step 2γ

### 4-Fluoro-2-(5-oxo-4,10-dioxa-tricyclo[5.2R.1R.0*2,6*]dec-3S-yl)-benzaldehyde

To a stirred solution of (4R,5R)-2-(2-Bromo-4-fluoro-phenyl)-3,4-dimethyl-5-phenyl-oxazolidine 22 (25.5 g, 72.8 mmol) in anhydrous THF (50 mL) at −78° C. and under a nitrogen atmosphere, was added slowly n-Butyl lithium 1.6M (32.9 ml, 82.2 mmol) keeping the internal temperature below −60° C. The resulting mixture was stirred for 10 minutes at −78° C., warmed up to −60° C. and stirred for 4 h.

At the same time, in a separate three neck 1 litre round bottle equipped with a condenser, dropping funnel and under a nitrogen atmosphere, 1,2-dibromoethane (7.95 ml, 92.2 mmol) was added slowly to a stirred suspension of magnesium (2.15 g, 92.2 mmol) in anhydrous THF (30 mL) maintaining constant reflux. Once the fizzing had stopped, anhydrous THF (100 ml) was added to suspend the white solid MgBr₂ and the suspension was cooled down to −60° C. To this cooled suspension, the lithium salt solution prepared above was added by cannula. The resulting mixture was warmed up to −15° C. and stirred for 30 minutes. Then, it was cooled down to −60° C. and a solution of norcantharidin (13.8 g, 82.2 mmol) in anhydrous THF (50 ml) was added dropwise over 15 minutes and the resulting solution was stirred for 30 minutes. After this time the mixture was warmed up to −30° C. and stirred for 2.5 h. Then the mixture was cooled down to −60° C. and quenched with methanol (100 ml), followed by portion wise addition of sodium borohydride (3.9 g, 101.9 mmol). The mixture was allowed to warm up to −25° C. and stirred for 1.5 h. A solution of hydrochloric acid (2M, 150 ml) was carefully added, the mixture was warmed up to room temperature and stirred for 14 h. The reaction mixture was concentrated in vacuo diluted with water (100 ml) and extracted with EtOAc (2×150 ml). The combined extracts were washed with brine (70 ml), dried over MgSO₄, filtered and the solvent was evaporated under vacuum to give crude product as a green solid (19.5 g). The crude product was purified by recrystallization from THF/isohexane (5:1) to yield the title compound as a white solid (10.1 g, yield=50%).

¹H-NMR (CDCl₃, 300 MHz) δ 10.05 (s, 1H, —CHO), 7.91 (dd, 1H, J=5.7, 8.6 Hz, ArH), 7.24 (m, 2H, ArH), 6.10 (d, 1H, J=3.1 Hz, —O—CH—Ar), 5.40 (m, 1H, —CH—O—), 4.97 (m, 1H, —CH—O), 2.87 (d, 1H, J=8.2 Hz, —CH—CO—), 2.28 (d, 1H, J=2.9, 8.2 Hz, —CH—), 1.84 (m, 2H, —CH₂—CH₂—), 1.55-1.44 (m, 2H, —CH₂—CH₂—). ¹⁹F-NMR (CDCl₃, 300 MHz) δ −110.6

### Step 3

### 3R-(5-Fluoro-2-hydroxymethyl-benzyl)-7-oxa-bicyclo[2.2.1]heptane-2R-carboxylic acid

A solution of 4-Fluoro-2-(5-oxo-4,10-dioxa-tricyclo[5.2R.1R.0*2,6*]dec-3S-yl)-benzaldehyde (20 g, 71.9 mmol) and Pd on alumina (10% reduced, 4 g) in ethanol (1000 ml) was stirred at RT under hydrogen atmosphere for 30 min. The reaction mixture was filtered through celite and concentrated in vacuo, redissolved in ethanol (1000 ml) and Pd on alumina (10% reduced, 4 g) was added. The mixture was stirred at RT under a hydrogen atmosphere for another 30 min, filtered through celite and concentrated in vacuo to yield the title compound as a white solid, (20 g, 99%).

¹H-NMR (CDCl₃, 300 MHz) δ 7.41 (m, 1H, ArH), 7.01 (m, 2H, ArH), 4.65 (s, 1H, —CH—O—), 4.46 (s, 2H, —O—CH₂—Ar), 4.01 (m, 1H, —CH—O—), 2.79 (d, 1H, —CHCO₂), 2.64-2.31 (m, 3H, —CHCH—O and —CH₂—Ar), 1.61-1.24 (m, 4H, —CH₂— and —CH₂—). ¹⁹F-NMR (CDCl₃, 300 MHz) δ −116.7.

LC-MS: m/z 281 M+H⁺

### Step 4

### 3R-(5-Fluoro-2-formyl-benzyl)-7-oxa-bicyclo[2.2.1]heptane-2R-carboxylic acid

To a solution of 3R-(5-Fluoro-2-hydroxymethyl-benzyl)-7-oxa-bicyclo[2.2.1]heptane-2R-carboxylic acid (20 g, 71 mmol) in refluxing DCE (700 mL) was added activated MnO₂ (67 g ml, 655 mmol) in 10 equal portions within 5 hours. Methanol (50 ml) was added, the mixture was allowed to cool to rt, filtered through a silica plug (2 cm), the solids were washed with isopropanol:MeOH (1:1, 1000 ml) and concentrated in vacuo to yield the title compound as a light brown solid (17.7 g, 90%).

LC-MS: m/z 279 M+H⁺. The aldehyde was used without further purification in the subsequent step.

### Step 5

### 3R-[5-Fluoro-2-(2-methoxycarbonyl-vinyl)-benzyl]-7-oxa-bicyclo[2.2.1]heptane-2R-carboxylic acid

To a solution of 3R-(5-Fluoro-2-formyl-benzyl)-7-oxa-bicyclo[2.2.1]heptane-2R-carboxylic acid. (15 g, 54 mmol) in THF (500 mL) at rt and under nitrogen atmosphere, (methoxycarbonylmethylene)triphenylphosphorane (27 g, 81 mmol) was added. The reaction mixture was stirred for 16 hours at rt before concentrating in vacuo. The residue was dissolved in DCM containing 10% of conc. NH₄OH:EtOAc (1:9) (150 ml). Triphenylphosphine oxide was removed from the crude product by filtering the ammonium salt through 500 g of silica. The title compound was washed out from silica using 10% AcOH in EtOAc and concentrated in vacuo to yield an off-white solid (13.2 g, 73%).

¹H-NMR (CDCl₃, 300 MHz) δ 7.91 (d, 1H, ═CH), 7.74 (broad s, 1H, —OH), 7.51 (dd, 1H, ArH), 6.92 (m, 2H, ArH), 6.28 (d, 1H, CH═CH), 4.87 (s, 1H, —CH—O), 4.11 (m, 1H, —CH—O—), 3.80 (s, 3H, —OCH₃), 2.94-2.73 (m, 1H, —CHCO₂, and 1H, —CH—CH₂—Ar), 2.65 (m, 1H, —CH—CHH—Ar 1H), 2.28 (m, 1H, —CH—CHH—Ar), 1.57 (m, 2H, —CH₂—), 1.40 (m, 1H, —CHH—), 1.25 (m, 1H, —CHH—). ¹⁹F-NMR (CDCl₃, 300 MHz) δ −110.4.

LC-MS: m/z 335 M+H⁺

### Step 6

### 3-(2R-{3R-[1-(4-Cyclohexyl-butylcarbamoyl)-2-hydroxy-ethylcarbamoyl]-7-oxa-bicyclo[2.2.1]hept-2-ylmethyl}-4-fluoro-phenyl)-acrylic acid methyl ester

To a solution of 3R-[5-Fluoro-2-(2-methoxycarbonyl-vinyl)-benzyl]-7-oxa-bicyclo[2.2.1]heptane-2R-carboxylic acid. (13.2 g, 39.5 mmol), 2-amino-N-(4-cyclohexyl-butyl)-3-hydroxy-propionamide (10.5 g, 43.5 mmol and NMM (6.8 ml, 59.3 mmol) in DCM (500 mL) with ice bath cooling, WSC HCl (11.4 g, 59.3 mmol) was added. After 30 minutes the ice bath was removed and the mixture was stirred for 16 hours at rt before concentrating in vacuo. The residue was dissolved in EtOAc washed with HCl (aq. 2M), sat. NaHCO₃ and brine. The extract was dried over MgSO₄, filtered and the solvent was evaporated under vacuum to give crude product as an off-white solid (12.5 g,).

¹H-NMR (CDCl₃, 300 MHz) δ 7.87 (d, 1H, ═CH), 7.55 (dd, 1H, ArH), 7.46 (d, 1H, —NH), 7.11-6.81 (m, 1H, —NH, 2H, ArH), 6.31 (d, 1H, CH═CH), 4.79 (m, 1H, —CH—O—), 4.50 (m, 1H, —CH—NH), 4.26 (m, 1H, —CH—O—), 4.03 (m, 2H, —CH₂OH), 3.81 (s, 3H, —CO₂CH₃), 3.70 (m, 1H, —OH), 3.12 (m, 2H, —CH₂—NH) 0.71-2.90 (m, 25H, —CH—+—CH₂—). ¹⁹F-NMR (CDCl₃, 300 MHz) δ −110.2.

LC-MS: m/z 559 M+H⁺

### Step 7

### (E)-3-(2R-{3R-[4-(4-Cyclohexyl-butylcarbamoyl)-4,5-dihydro-oxazol-2-yl]-7-oxa-bicyclo[2.2.1]hept-2-ylmethyl}-4-fluoro-phenyl)-acrylic acid methyl ester

(E)-3R-(2R-{3-[1-(4-Cyclohexyl-butylcarbamoyl)-2-hydroxy-ethylcarbamoyl)-2-hydroxy-ethylcarbamoyl]-7-oxa-bicyclo[2.2.1]hept-2-ylmethyl}-4-fluoro-phenyl)-acrylic acid methyl ester (61) (12.5 g, 22 mmol) was dissolved in dry DCM (1000 mL) at −78° C., under N₂. DAST (6.1 mL, 44 mmol) was added and the reaction mixture was stirred for two hours at −78° C. K₂CO₃ (12.2 g, 88 mmol) was added and the mixture was stirred overnight at room temperature. NaHCO₃ saturated solution and DCM were added. The organic phase was separated, washed with brine, dried over MgSO₄, filtered and concentrated under vacuum. The desired product was purified by recrystallisation from diethyl ether to yield an off-white solid (11.0 g, 93% yield).

¹H-NMR (CDCl₃, 300 MHz) δ 7.89(d, 1H, J=16 Hz, —CH═CO₂Me), 7.60 (m, 1H, ArH), 6.98 (m, 2H, ArH), 6.86 (m, 1H, NH), 6.35 (d, 1H, J=16 Hz, —CH═Ar), 4.86 (m, 1H, —CH—O—), 4.71(m, 1H, —N—CH—CH₂—O), 4.51 (m, 2H, —N—CH—CH₂—O), 4.33 (m, 1H, —CH—O—), 3.85 (s, 3H, —CO₂CH₃), 0.84-3.23 (m, 27H, —CH—+—CH₂—). ¹⁹F-NMR (CDCl₃, 300 MHz) δ −110.2.

LC-MS: m/z 541 M+H^{|}

### Step 8

### (E)-3-(2R-{3R-[4-(4-Cyclohexyl-butylcarbamoyl)-oxazol-2-yl]-7-oxa-bicyclo[2.2.1]hept-2-ylmethyl}-4-fluoro-phenyl)-acrylic acid methyl ester

To a solution of CuBr₂ (18.2 g, 81.4 mmol) in dry degassed DCM (350 mL), HMTA (11.4 g, 81.4 mmol) and DBU (12.2 ml, 81.4 mmol) were added at 0° C. and stirred for 10 minutes. (E)-3R-(2R-{3-[4-(4-Cyclohexyl-butylcarbamoyl)-4,5-dihydro-oxazol-2-yl]-7-oxa-bicyclo[2.2.1]hept-2-ylmethyl}-4-fluoro-phenyl)-acrylic acid methyl ester (62) (11 g, 20.3 mmol) in dry degassed DCM (150 mL), was added to the mixture which was stirred overnight at room temperature. The reaction mixture was concentrated in vacuo, suspended in a 1:1 solution of ammonia 33% and saturated solution of NH₄Cl (400 ml), extracted with EtOAc, washed with 1M HCl (400 ml) then sat. NaHCO₃ (400 ml) and finally brine (400 ml). The organic layer was dried over MgSO₄, filtered, and concentrated in vacuo to give a crude product which was purified by recrystallization from diethyl ether (10.5 g, 85% yield).

¹H-NMR (CDCl₃, 300 MHz) δ 8.10 (s, 1H, O—CH═C—N), 7.86 (d, 1H, J=16 Hz, —CH═CO₂Me), 7.56 (m, 1H, ArH), 7.14 (m, 1H, ArH), 6.91 (m, 1H, ArH), 6.32 (d, 1H, J=16 Hz, —CH═Ar), 5.00 (m, 1H, —CH—O—), 4.37 (m, 1H, —CH—O—), 3.85 (s, 3H, —CO₂CH₃), 3.41 (m, 3H, NH—CH₂+N═C—CH), 3.07 (m, 1H, CH—CH₂—Ar), 2.43 (m, 2H, CH₂—Ar), 0.85-1.85 (m, 21H, —CH—+—CH₂—). ¹⁹F-NMR (CDCl₃, 300 MHz) δ −110.6.

LC-MS: m/z 539 M+H⁺

### Step 9

### ((E)-3-(2R-{3R-[4-(4-Cyclohexyl-butylcarbamoyl)-oxazol-2-yl]-7-oxa-bicyclo[2.2.1]hept-2-ylmethyl}-4-fluoro-phenyl)-acrylic acid

(E)-3R-(2R-{3-[4-(4-Cyclohexyl-butylcarbamoyl)-oxazol-2-yl]-7-oxa-bicyclo[2.2.1]hept-2-ylmethyl}-4-fluoro-phenyl)-acrylic acid methyl ester (5.5 g, 10.2 mmol) was dissolved in THF (30 mL), MeOH (165 ml) and 1M NaOH (65 ml, 65 mmol) was added. The mixture stirred 5 hours at room temperature, acidified with 1M HCl (100 ml, 100 mmol) extracted with DCM (2×200 ml) dried over MgSO₄, filtered and concentrated in vacuo. The crude product was recrystallized from diethyl ether to yield the titled compound as a white solid. (4.9 g, 92% yield).

¹H-NMR (CDCl₃, 300 MHz) δ 8.19 (s, 1H, O—CH═C—N), 7.92 (d, 1H, J=16 Hz, —CH═CO₂Me), 7.58 (m, 1H, ArH), 7.14 (m, 1H, ArH), 6.93 (m, 1H, ArH), 6.33 (d, 1H, J=16 Hz, —CH═Ar), 5.01 (m, 1H, —CH—O—), 4.39 (m, 1H, —CH—O—), 3.41 (m, 4H, NH—CH₂+N═C—CH+CH—CH₂—Ar), 0.83-2.57 (m, 24H, —CH—+—CH₂—). ¹⁹F-NMR (CDCl₃, 300 MHz) δ −110.0.

LC-MS: m/z 525 M+H⁺

### General Method:

The compound of Example 1 (0.06 g, 0.114 mmol) was dissolved in dichloromethane (DCM) (2.5 mL) and cooled at 0° C. Carbonyldimidazole (0.022 g, 1.37 mmol), was added and the reaction mixture was stirred for 30 minutes. The sulfonylamide (0.024 g, 0.23 mmol) and diazabicycloundec-7-ene (DBU) (0.019 mL, 0.13 mmol) were added and the reaction mixture was stirred overnight at room temperature. Dichloromethane (15 mL) was added and the mixture was washed with 1M HCl, dried over MgSO₄, filtered and concentrated “under vacuum”.

The crude product was purified by column chromatography on a 1 g SPE cartridge, using a solvent gradient using dichloromethane to dichloromethane/methanol 100:3 to isolate the title compound as a solid.

### EXAMPLE 2

### (E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(ethylsulfonyl)acrylic amide

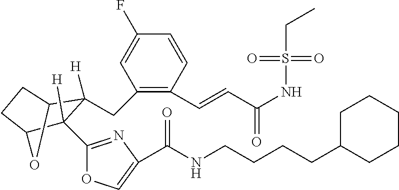 [SMILES: CCS(=O)(=O)NC(=O)/C=C/c1ccc(F)cc1CC1C2CCC(O2)C1c1nc(C(=O)NCCCCC2CCCCC2)co1]

This compound was prepared following general method 1 and using ethanesulfonamide as the reagent. Yield: 70%

¹H-NMR (CDCl₃, 300 MHz) δ 8.13 (s, 1H, ═CH—O—), 7.80 (d, 1H, J=16 Hz, —CH═), 7.55 (dd, 1H, J=5.7, 8.6 Hz, ArH), 6.93 (m, 2H, ArH), 6.31 (d, 1H, J=16 Hz, —CH═), 5.10 (m, 1H, —CH—O—), 4.28 (m, 1H, —CH—O), 3.59 (m, 2H, —S—CH₂—CH₃), 3.43(m, 4H, —CH—+—CH₂), 2.44(m, 2H, —NH—CH₂—), 1.80-0.84 (m, 24H, —CH—+—CH₂—+—CH₃).

¹⁹F-NMR (CDCl₃, 300 MHz) δ −110.

LC-MS: m/z 616 M+H⁺

### EXAMPLE 3

### (E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(methylsulfonyl)acrylic amide

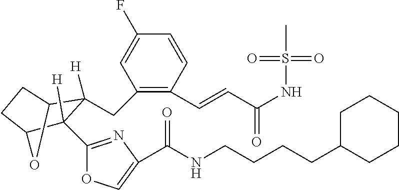 [SMILES: CS(=O)(=O)NC(=O)/C=C/c1ccc(F)cc1CC1C2CCC(O2)C1c1nc(C(=O)NCCCCC2CCCCC2)co1]

This compound was prepared following general method 1 and using methanesulfonamide as the reagent. Yield: 65%

¹H-NMR (CDCl₃, 300 MHz) δ 8.13 (s, 1H, ═CH—O—), 7.80 (d, 1H, J=16 Hz, —CH═), 7.55 (dd, 1H, J=5.7, 8.6 Hz, ArH), 6.93 (m, 2H, ArH), 6.31 (d, 1H, J=16 Hz, —CH═), 5.10 (m, 1H, —CH—O—), 4.28 (m, 1H, —CH—O), 3.65 (s, 3H, —S—CH₃), 3.43 (m, 4H, —CH—+—CH₂), 2.44 (m, 2H, —NH—CH₂—), 1.80-0.84 (m, 21H, —CH—+—CH₂—).

¹⁹F-NMR (CDCl₃, 300 MHz) δ −110.5.

LC-MS: m/z 602 M+H⁺

### EXAMPLE 4

### (E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2,2,2-trifluoroethanesulfonyl)acrylic amide

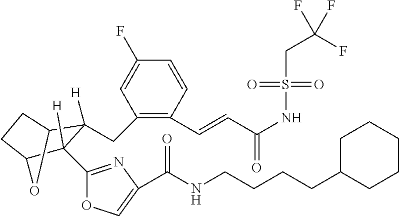 [SMILES: O=C(/C=C/c1ccc(F)cc1CC1C2CCC(O2)C1c1nc(C(=O)NCCCCC2CCCCC2)co1)NS(=O)(=O)CC(F)(F)F]

This compound was prepared following general method 1 and using 2,2,2-trifluoroethanesulfonamide as the reagent. Yield: 57%

¹H-NMR (CDCl₃, 300 MHz) δ 8.20 (d, 1H, J=16 Hz, —CH═), 8.10 (s, 1H, ═CH—O—), 7.55 (dd, 1H, J=5.7, 8.6 Hz, ArH), 6.93 (m, 2H, ArH), 6.70 (d, 1H, J=16 Hz, —CH═), 5.05 (m, 1H, —CH—O—), 4.40 (m, 1H, —CH—O), 3.95 (m, 2H, —S—CH₂—CF₃), 3.40 (m, 2H, —CH₂Ar), 3.30 (m, 2H, —NH—CH₂—), 1.80-0.84 (m, 23H, —CH—+—CH₂—).

¹⁹F-NMR (CDCl₃, 300 MHz) δ −63 and −107.

LC-MS: m/z 670 M+H⁺

### EXAMPLE 5

### (E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2-methylethanesulfonyl)acrylic amide

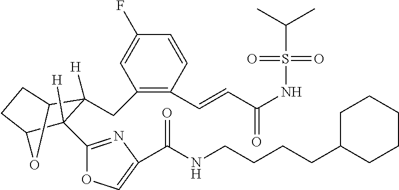 [SMILES: CC(C)S(=O)(=O)NC(=O)/C=C/c1ccc(F)cc1CC1C2CCC(O2)C1c1nc(C(=O)NCCCCC2CCCCC2)co1]

This compound was prepared following general method 1 and using 2-methylethanesulfonamide as the reagent. Yield: 70%

¹H-NMR (CDCl₃, 300 MHz) δ 8.13 (s, 1H, ═CH—O—), 7.80 (d, 1H, J=16 Hz, —CH═), 7.55 (dd, 1H, J=5.7, 8.6 Hz, ArH), 6.93 (m, 2H, ArH), 6.31 (d, 1H, J=16 Hz, —CH═), 5.10 (m, 1H, —CH—O—), 4.28 (m, 1H, —CH—O), 3.59 (m, 2H, —S—CH₂—CH₃), 3.43(m, 4H, —CH—+—CH₂), 2.44(m, 2H, —NH—CH₂—), 1.80-0.84 (m, 24H, —CH—+—CH₂—+—CH₃).

¹⁹F-NMR (CDCl₃, 300 MHz) δ −110.7.

LC-MS: m/z 630 M+H⁺

### EXAMPLE 6

### (E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(benzenesulfonyl)acrylic amide

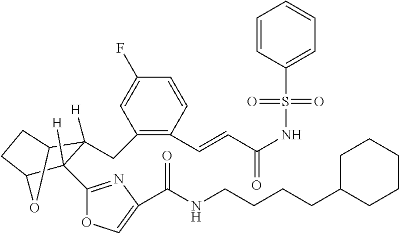 [SMILES: O=C(/C=C/c1ccc(F)cc1CC1C2CCC(O2)C1c1nc(C(=O)NCCCCC2CCCCC2)co1)NS(=O)(=O)c1ccccc1]

This compound was prepared following general method 1 and using benzenesulfonamide as the reagent. Yield: 75%

¹H-NMR (CDCl₃, 300 MHz) δ 8.13 (s, 1H, ═CH—O—), 7.80 (d, 1H, J=16 Hz, —CH═), 7.55 (dd, 1H, J=5.7, 8.6 Hz, ArH), 7.33 (m, 5H, —SArH), 6.93 (m, 2H, ArH), 6.31 (d, 1H, J=16 Hz, —CH═), 5.10 (m, 1H, —CH—O—), 4.28 (m, 1H, —CH—O), 3.43(m, 4H, —CH—+—CH₂), 2.44(m, 2H, —NH—CH₂—), 1.80-0.84 (m, 21H, —CH—+—CH₂—+—CH₃).

¹⁹F-NMR (CDCl₃, 300 MHz) δ −110.1.

LC-MS: m/z 664 M+H⁺

### EXAMPLE 7

### (E)-3R-[2R-[[3-[4-[[(octylamino]carbonyl]-2-oxazolyl]-7-oxabicyclo[2.2.1]hept-2-yl]methyl]-4-fluoro-phenyl]-N-(ethylsulfonyl)acrylic amide

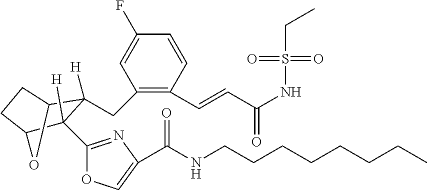 [SMILES: CCCCCCCCNC(=O)c1coc(C2C3CCC(O3)C2Cc2cc(F)ccc2/C=C/C(=O)NS(=O)(=O)CC)n1]

### Step 1

### 3-(2R-{3R-[1-Octylcarbamoyl-2-hydroxy-ethylcarbamoyl]-7-oxa-bicyclo[2.2.1]hept-2-ylmethyl}-4-fluoro-phenyl)-acrylic acid methyl ester

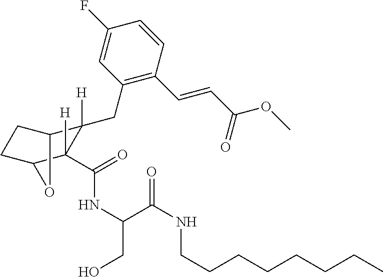 [SMILES: CCCCCCCCNC(=O)C(CO)NC(=O)C1C2CCC(O2)C1Cc1cc(F)ccc1/C=C/C(=O)OC]

To a solution of 3R-[5-Fluoro-2-(2-methoxycarbonyl-vinyl)-benzyl]-7-oxa-bicyclo[2.2.1]heptane-2R-carboxylic acid. (13.2 g, 39.5 mmol), 2-amino-N-octyl-3-hydroxy-propionamide (10.5 g, 43.5 mmol and NMM (6.8 ml, 59.3 mmol) in DCM (500 mL) with ice bath cooling, N-(3-dimethylaminopropyl) N′-ethylcarbodiimide hydrochloride (WSC HCl) (11.4 g, 59.3 mmol) was added. After 30 minutes the ice bath was removed and the mixture was stirred for 16 hours at room temperature before concentrating in vacuo. The residue was dissolved in ethylacetate (EtOAc) washed with HCl (aq. 2M), sat. NaHCO₃ and brine. The extract was dried over MgSO₄, filtered and the solvent was evaporated under vacuum to give crude product as an off-white solid.

¹H-NMR (CDCl₃, 300 MHz) δ 7.87 (d, 1H, ═CH), 7.55 (dd, 1H, ArH), 7.46 (d, 1H, —NH), 7.11-6.81 (m, 1H, —NH, 2H, ArH), 6.31 (d, 1H, CH═CH), 4.79 (m, 1H, —CH—O—), 4.50 (m, 1H, —CH—NH), 4.26 (m, 1H, —CH—O—), 4.03 (m, 2H, —CH₂OH), 3.81 (s, 3H, —CO₂CH₃), 3.70 (m, 1H, —OH), 3.12 (m, 2H, —CH₂—NH), 2.90-0.71 (m, 21H, —CH₂—+—CH₃).

¹⁹F-NMR (CDCl₃, 300 MHz) δ −110.2.

### Step 2

### (E)-3-(2R-{3R-[4-(Octylcarbamoyl)-4,5-dihydro-oxazol-2-yl]-7-oxa-bicyclo[2.2.1]hept-2-ylmethyl}-4-fluoro-phenyl)-acrylic acid methyl ester

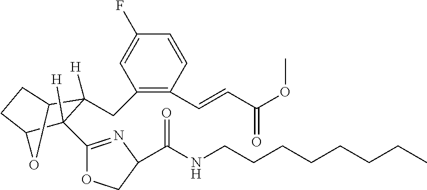 [SMILES: CCCCCCCCNC(=O)C1COC(C2C3CCC(O3)C2Cc2cc(F)ccc2/C=C/C(=O)OC)=N1]

(E)-3R-(2R-{3-[1-(octylcarbamoyl)-2-hydroxy-ethylcarbamoyl)-2-hydroxy-ethylcarbamoyl]-7-oxa-bicyclo[2.2.1]hept-2-ylmethyl}-4-fluoro-phenyl)-acrylic acid methyl ester (61) (12.5 g, 22 mmol) was dissolved in dry DCM (1000 mL) at −78° C., under N₂. N,N-Diethylamino sulfur trifluoride (DAST) (6.1 mL, 44 mmol) was added and the reaction mixture was stirred for two hours at −78° C. K₂CO₃ (12.2 g, 88 mmol) was added and the mixture was stirred overnight at room temperature. NaHCO₃ saturated solution and DCM were added. The organic phase was separated, washed with brine, dried over MgSO₄, filtered and concentrated under vacuum. The desired product was purified by recrystallisation from diethyl ether to yield an off-white solid (11.0 g, 93% yield).

¹H-NMR (CDCl₃, 300 MHz) δ 7.89(d, 1H, J=16 Hz, —CH═CO₂Me), 7.60 (m, 1H, ArH), 6.98 (m, 2H, ArH), 6.86 (m, 1H, NH), 6.35 (d, 1H, J=16 Hz, —CH═Ar), 4.86 (m, 1H, —CH—O—), 4.71(m, 1H, —N—CH—CH₂—O), 4.51 (m, 2H, —N—CH-CH₂—O), 4.33 (m, 1H, —CH—O—), 3.85 (s, 3H, —CO₂CH₃), 3.23-0.84 (m, 21H, —CH₂+—CH₃).

¹⁹F-NMR (CDCl₃, 300 MHz) δ −110.2.

### Step 3

### (E)-3-(2R-{3R-[4-(Octylbutylcarbamoyl)-oxazol-2-yl]-7-oxa-bicyclo[2.2.1]hept-2-ylmethyl}-4-fluoro-phenyl)-acrylic acid methyl ester

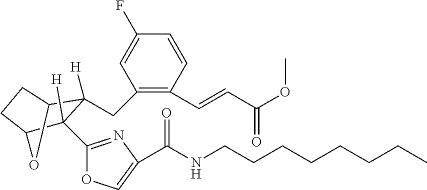 [SMILES: CCCCCCCCNC(=O)c1coc(C2C3CCC(O3)C2Cc2cc(F)ccc2/C=C/C(=O)OC)n1]

To a solution of CuBr₂ (18.2 g, 81.4 mmol) in dry degassed DCM (350 mL), hexamethylene tetraamine (HMTA) (11.4 g, 81.4 mmol) and DBU (12.2 ml, 81.4 mmol) were added at 0° C. and stirred for 10 minutes. (E)-3R-(2R-{3-[4-(octylcarbamoyl)-4,5-dihydro-oxazol-2-yl]-7-oxa-bicyclo[2.2.1]hept-2-ylmethyl}-4-fluoro-phenyl)-acrylic acid methyl ester (11 g, 20.3 mmol) in dry degassed DCM (150 mL), was added to the mixture which was stirred overnight at room temperature. The reaction mixture was concentrated in vacuo, suspended in a 1:1 solution of ammonia 33% and saturated solution of NH₄Cl (400 ml), extracted with EtOAc, washed with 1M HCl (400 ml) then sat. NaHCO₃ (400 ml) and finally brine (400 ml). The organic layer was dried over MgSO₄, filtered, and concentrated in vacuo to give a crude product which was purified by recrystallization from diethyl ether. (10.5 g, 85% yield).

¹H-NMR (CDCl₃, 300 MHz) δ 8.10 (s, 1H, O—CH═C—N), 7.86 (d, 1H, J=16 Hz, —CH═CO₂Me), 7.56 (m, 1H, ArH), 7.14 (m, 1H, ArH), 6.91 (m, 1H, ArH), 6.32 (d, 1H, J=16 Hz, —CH═Ar), 5.00 (m, 1H, —CH—O—), 4.37 (m, 1H, —CH—O—), 3.85 (s, 3H, —CO₂CH₃), 3.41 (m, 3H, NH—CH₂+N═C—CH), 3.07 (m, 1H, CH—CH₂—Ar), 2.43 (m, 2H, CH₂—Ar), 1.85-0.85 (m, 18H, —CH₂—+CH₃).

¹⁹F-NMR (CDCl₃, 300 MHz) δ −110.6.

### Step 4

### ((E)-3-(2R-{3R-[4-(Octylcarbamoyl)-oxazol-2-yl]-7-oxa-bicyclo[2.2.1]hept-2-ylmethyl}-4-fluoro-phenyl)-acrylic acid

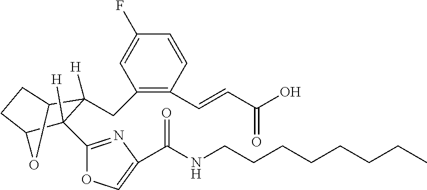 [SMILES: CCCCCCCCNC(=O)c1coc(C2C3CCC(O3)C2Cc2cc(F)ccc2/C=C/C(=O)O)n1]

(E)-3R-(2R-{3-[4-(octylcarbamoyl)-oxazol-2-yl]-7-oxa-bicyclo[2.2.1]hept-2-ylmethyl}-4-fluoro-phenyl)-acrylic acid methyl ester (5.5 g, 10.2 mmol) was dissolved in tetrahydrofuran (THF) (30 mL), methanol (MeOH) (165 ml) and 1M NaOH (65 ml, 65 mmol) was added. The mixture stirred 5 hours at room temperature, acidified with 1M HCl (100 ml, 100 mmol) extracted with DCM (2×200 ml) dried over MgSO₄, filtered and concentrated in vacuo. The crude product was recrystallized from diethyl ether to yield the title compound as a white solid. (4.9 g, 92% yield).

¹H-NMR (CDCl₃, 300 MHz) δ 8.19 (s, 1H, O—CH═C—N), 7.92 (d, 1H, J=16 Hz, —CH═CO₂Me), 7.58 (m, 1H, ArH), 7.14 (m, 1H, ArH), 6.93 (m, 1H, ArH), 6.33 (d, 1H, J=16 Hz, —CH═Ar), 5.01 (m, 1H, —CH—O—), 4.39 (m, 1H, —CH—O—), 3.41 (m, 4H, NH—CH₂+N═C—CH+CH—CH₂—Ar), 2.57-0.83 (m, 21H, —CH₂—+CH₃).

¹⁹F-NMR (CDCl₃, 300 MHz) δ −110.0.

### Step 5

### (E)-3R-[2R-[[3-[4-[[(octylamino]carbonyl]-2-oxazolyl]-7-oxabicyclo[2.2.1]hept-2-yl]methyl]-4-fluoro-phenyl]-N-(ethylsulfonyl)acrylic amide

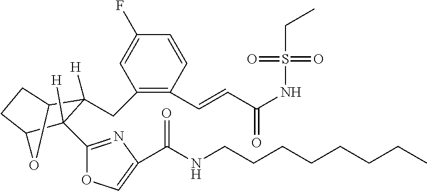 [SMILES: CCCCCCCCNC(=O)c1coc(C2C3CCC(O3)C2Cc2cc(F)ccc2/C=C/C(=O)NS(=O)(=O)CC)n1]

This compound was prepared from the product of Example 7, Step 4, following general method 1 and using ethanesulfonamide as the reagent. Yield: 70%

¹H-NMR (CDCl₃, 300 MHz) δ 8.13 (s, 1H, ═CH—O—), 7.80 (d, 1H, J=16 Hz, —CH═), 7.55 (dd, 1H, J=5.7, 8.6 Hz, ArH), 6.93 (m, 2H, ArH), 6.31 (d, 1H, J=16 Hz, —CH═), 5.10 (m, 1H, —CH—O—), 4.28 (m, 1H, —CH—O), 3.59 (m, 2H, —S—CH₂—CH₃), 3.43(m, 4H, —CH—+—CH₂), 2.44(m, 2H, —NH—CH₂—), 1.80-0.84 (m, 19H, —CH₂—+—CH₃).

¹⁹F-NMR (CDCl₃, 300 MHz) δ −110.0.

LC-MS: m/z 591 M+H⁺

### EXAMPLE 8

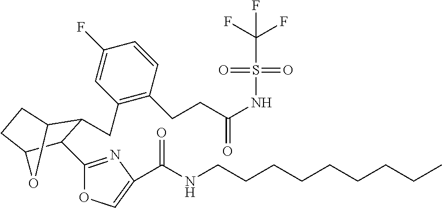 [SMILES: CCCCCCCCCNC(=O)c1coc(C2C3CCC(O3)C2Cc2cc(F)ccc2CCC(=O)NS(=O)(=O)C(F)(F)F)n1]

### Step 1

### (4R,5R)-2-(2-Bromo-4-fluoro-phenyl)-3,4-dimethyl-5-phenyl-oxazolidine

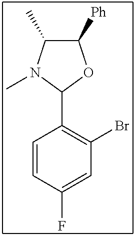 [SMILES: C[C@@H]1[C@@H](c2ccccc2)OC(c2ccc(F)cc2Br)N1C]

To a solution 2-bromo-4-fluorobenzaldehyde (15.2 g, 74.9 mmol) in toluene (80 ml) was added (1R,2R)-(−)-pseudoephedrine (13.6 g, 82 mmol) and the resulting mixture was refluxed removing water with Dean-Stark trap for 16 h. The reaction was stopped and cooled down to room temperature. The solution was washed with citric acid solution (1M, 100 ml), saturated solution of sodium bicarbonate (50 ml), brine (50 ml) and dried (MgSO₄). Then, it was filtered and the solvent was evaporated under vacuum to give title compound as a yellow oil. (26.2, yield=97%).

¹H-NMR (CDCl₃, 300 MHz) δ 7.78 (dd, 1H, J=5.7, 8.6 Hz, ArH), 7.36 (m, 6H, ArH), 7.11 (m, 1H, ArH), 5.47 (s, 1H, —N—CH—O—), 4.71 (d, 1H, J=8.64 Hz, —CH-Ph), 2.60 (m, 1H, —CH—CH₃), 2.27 (s, 3H, —CHCH₃). ¹⁹F-NMR (CDCl₃, 300 MHz) δ −111.6

### Step 2

### 4-Fluoro-2-(5-oxo-4,10-dioxa-tricyclo[5.2.1.0*2,6*]dec-3-yl)-benzaldehyde

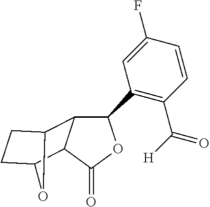 [SMILES: O=Cc1ccc(F)cc1[C@H]1OC(=O)C2C3CCC(O3)C21]

To a stirred solution of (4R,5R)-2-(2-Bromo-4-fluoro-phenyl)-3,4-dimethyl-5-phenyl-oxazolidine (25.5 g, 72.8 mmol) in anhydrous THF (50 mL) at −78° C. and under nitrogen atmosphere, was added slowly n-Butyl lithium 1.6M (32.9 ml, 82.2 mmol) keeping the internal temperature below −60° C. The resulting mixture was stirred for 10 minutes at −78° C., warmed up to −60° C. and stirred for 4 h.

At the same time, in a separate three neck 1 litre round bottle equipped with condenser, dropping funnel and under nitrogen atmosphere, 1,2-dibromoethane (7.95 ml, 92.2 mmol) was added slowly to a stirred suspension of magnesium (2.15 g, 92.2 mmol) in anhydrous THF (30 mL) maintaining constant reflux. Once the fizzing was stopped, anhydrous THF (100 ml) was added to suspend the white solid MgBr₂ and the suspension cooled down to −60° C. To this cooled suspension, the lithium salt solution prepared above was added by canulation. The resulting mixture was warmed up to −15° C. and stirred for 30 minutes.

Then, it was cooled down to −60° C. and a solution of norcantharidin (13.8 g, 82.2 mmol) in anhydrous THF (50 ml) was added drop wise under 15 minutes and the resulting solution stirred for 30 minutes. After, the mixture was warmed up to −30° C. and stirred for 2.5 h.

Then the mixture was cooled down to −60° C. and quenched with methanol (100 ml), followed by a portion wise addition (20) sodium borohydride (3.9 g, 101.9 mmol). The mixture was let to warm up to −25° C. and stirred for 1.5 h.

A solution of hydrochloric acid (2M, 150 ml) was carefully added, the mixture was warmed up to room temperature and stirred for 14 h. The reaction mixture was concentrated in vacuo diluted with water (100 ml) and extracted with EtOAc (2×150 ml). The combined extracts were washed with brine (70 ml), dried over MgSO₄, filtered and the solvent was evaporated under vacuum to give the crude product as a green solid (19.5 g).

The crude product was purified by crystallization from THF/isohexane (5:1) to yield the titled compound as a white solid (10.1 g, yield=50%).

¹H-NMR (CDCl₃, 300 MHz) δ 10.05 (s, 1H, —CHO), 7.91 (dd, 1H, J=5.7, 8.6 Hz, ArH), 7.24 (m, 2H, ArH), 6.10 (d, 1H, J=3.1 Hz, —O—CH—Ar), 5.40 (m, 1H, —CH—O—), 4.97 (m, 1H, —CH—O), 2.87 (d, 1H, J=8.2 Hz, —CH—CO—), 2.28 (d, 1H, J=2.9, 8.2 Hz, —CH—), 1.84 (m, 2H, —CH₂—CH₂—), 1.55-1.44 (m, 2H, —CH₂—CH₂—). ¹⁹F-NMR (CDCl₃, 300 MHz) δ −110.6

### Step 3

### (E)-3-[4-Fluoro-2-((S)-5-oxo-4,10-dioxa-tricyclo[5.2.1.0*2,6*]dec-3-yl)-phenyl]-acrylic acid methyl ester

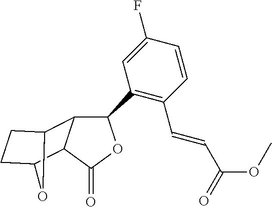 [SMILES: COC(=O)/C=C/c1ccc(F)cc1[C@H]1OC(=O)C2C3CCC(O3)C21]

To a solution of 4-Fluoro-2-(5-oxo-4,10-dioxa-tricyclo[5.2.1.0*2,6*]dec-3-yl)-benzaldehyde (5 g, 18.1 mmol) and lithium chloride (0.921 g, 21.72 mmol) in acetonitrile (30 ml) under nitrogen atmosphere, trimethylphosphonoacetate (3.13 mL, 21.72 mmol) was added followed by DBU (6.5 mL, 43.44 mmol). The resulting mixture was stirred at room temperature for 2 h. After this time it was poured over saturated solution of NaHCO₃ (100 mL) and extracted with dichloromethane (2×100 mL). The combined organic layers were dried over MgSO₄, filtered and concentrated in vacuo yielding the titled compound as a thick oil.

### Step 4

### 3-[5-Fluoro-2-(2-methoxycarbonyl-ethyl)-benzyl]-7-oxa-bicyclo[2.2.1]heptane-2-carboxylic acid

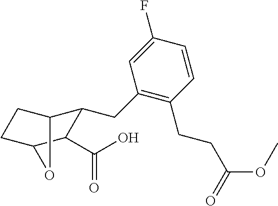 [SMILES: COC(=O)CCc1ccc(F)cc1CC1C2CCC(O2)C1C(=O)O]

To a solution of (E)-3-[4-Fluoro-2-((S)-5-oxo-4,10-dioxa-tricyclo[5.2.1.0*2,6*]dec-3-yl)-phenyl]-acrylic acid methyl ester (18.1 mmol) in a mixture 2:1 methanol/tetrahydrofuran (75 ml), palladium hydroxide (0.61 g) was added. The flask was evacuated and then connected to a balloon filled with hydrogen. The reaction was stirred at room temperature for 2 h, and a second portion of palladium hydroxide (0.61 g) was added. The flask was evacuated and then connected to a balloon filled with hydrogen. After another 2 h, the balloon was removed and Celite (1 g) was added to the mixture, which was stirred for 10 minutes. The mixture was filtered through a Celite pad and the pad was washed with methanol (25 mL). The filtrate was evaporated to provide a yellow oil. The oil was dissolved in dichloromethane (50 mL) and dried over MgSO₄.

Then, the solution was filtered and concentrated in vacuo and the residue was dissolved in ethyl acetate (60 mL) and treated with Darco KB activated carbon by heating at reflux for 2 minutes and then cooling. Celite (1.2 g) was added and the mixture stirred for 10 minutes and then filtered through a pad of Celite. The pad was washed with ethyl acetate (25 mL). The filtrate was evaporated and the residue was crystallized from hot ethyl acetate (11.5 mL) and heptane (23 mL). After cooling to room temperature, additional heptane (30 mL) was added and the mixture was left to stand at 4° C. overnight.

The solid was filtered and washed with more heptane and dried under vacuo overnight yielding the titled compound as a colorless solid. (5.36 g, 88%)

¹H-NMR (CDCl₃, 300 MHz) δ 7.11 (dd, 1H, J=5.7, 8.6 Hz, ArH), 6.88 (m, 2H, ArH), 4.88 (m, 1H, —CH—O—), 4.27 (m, 1H, —CH—O), 3.66 (s, 3H, CO₂CH₃), 2.92 (m, 3H, —CH₂—CH₂—CO₂Me and —CH—CO₂H), 2.72 (m, 1H, —CH—), 2.50 (m, 4H, —CH₂—CH₂—CO₂Me and —CH₂—Ar), 1.74 (m, 2H, —CH₂—CH₂—), 1.54-1.25 (m, 2H, —CH₂—CH₂—).

### Step 5

### 3-{4-Fluoro-2-[3-(2-hydroxy-1-nonylcarbamoyl-ethylcarbamoyl)-7-oxa-bicyclo[2.2.1]hept-2-ylmethyl]-phenyl}-propionic acid methyl ester

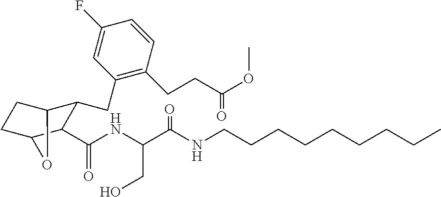 [SMILES: CCCCCCCCCNC(=O)C(CO)NC(=O)C1C2CCC(O2)C1Cc1cc(F)ccc1CCC(=O)OC]

To a solution of 3-[5-Fluoro-2-(2-methoxycarbonyl-ethyl)-benzyl]-7-oxa-bicyclo[2.2.1]heptane-2-carboxylic acid (5 g, 14.86 mmol) and nonylserinamide (3.77 g, 16.35 mmol) in dimethylformamide (150 ml) under nitrogen atmosphere, N-methylmorpholine (3.6 mL, 32.7 mmol) was added followed by HBTU (6.2 g, 16.35 mmol). The resulting mixture was stirred at room temperature for 16 h.

After this time the solution was concentrated under vacuum and the residue was dissolved in ethyl acetate (100 mL). Then, it was washed with a 2M HCl solution (100 mL), saturated solution of NaHCO₃ (100 mL) and dried over MgSO₄. Filtration and concentrated in vacuo yield the titled compound as a thick oil.

LC-MS: m/z 549 M+H⁺

### Step 6

### 3-{4-Fluoro-2-[3-(4-nonylcarbamoyl-4,5-dihydro-oxazol-2-yl)-7-oxa-bicyclo[2.2.1]hept-2-ylmethyl]-phenyl}-propionic acid methyl ester

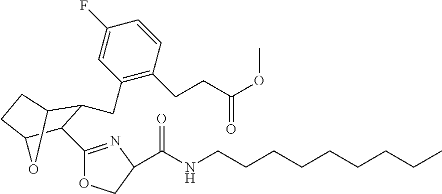 [SMILES: CCCCCCCCCNC(=O)C1COC(C2C3CCC(O3)C2Cc2cc(F)ccc2CCC(=O)OC)=N1]

To a solution of 3-{4-Fluoro-2-[3-(2-hydroxy-1-nonylcarbamoyl-ethylcarbamoyl)-7-oxa-bicyclo[2.2.1]hept-2-ylmethyl]-phenyl}-propionic acid methyl ester (14.86 mmol) in dichloromethane (200 ml), at −78° C. and under nitrogen atmosphere, DAST (3.93 mL, 29.72 mmol) was added and the resulting mixture was stirred at room temperature for 2.5 h.

After this time, potassium carbonate (4.11 g, 29.72 mmol) was added and the solution was stirred for another hour. Then saturated solution of NaHCO₃ (200 mL) was added and the mixture was extracted with ethyl acetate (200 mL). Then, it was washed with brine (150 mL), and dried over MgSO₄. Filtration and concentrated in vacuo yield the crude compound as a thick oil.

The residue was purified by column chromatography in silica using a solvent gradient starting from Ethyl acetate/iso-hexane 1:1 to Ethyl acetate/methanol 9:1 to isolate the titled compound as a thick oil (4.7 g, 60%)

¹H-NMR (CDCl₃, 300 MHz) δ 7.11 (dd, 1H, J=5.7, 8.6 Hz, ArH), 6.87 (m, 2H, ArH), 6.63 (m, 1H, NH), 4.87 (m, 1H, —CH—O—), 4.59 (m, 1H, ═N—CH—CON—), 4.40 (m, 2H, O—CH₂—), 4.31 (m, 1H, —CH—O), 3.67 (s, 3H, CO₂CH₃), 3.23 (m, 2H, —CONH—CH₂—), 2.94 (m, 3H, —CH₂—CH₂—CO₂Me and —CH—), 2.65-2.40 (m, 5H, —CH₂—CH₂—CO₂Me, —CH— and —CH₂—Ar), 1.76 (m, 2H, —CH₂—CH₂—), 1.55-1.44 (m, 4H, —CH₂—CH₂— and —CO—NH—CH₂—CH₂—), 1.27 (m, 12H, —CH₂—CH₂—), 0.88 (m, 3H, —CH₃).

LC-MS: m/z 531 M+H⁺

### Step 7

### 3-{4-Fluoro-2-[3-(4-nonylcarbamoyl-oxazol-2-yl)-7-oxa-bicyclo[2.2.1]hept-2-ylmethyl]-phenyl}-propionic acid methyl ester

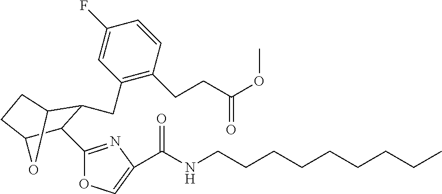 [SMILES: CCCCCCCCCNC(=O)c1coc(C2C3CCC(O3)C2Cc2cc(F)ccc2CCC(=O)OC)n1]

To a suspension of Copper bromide (6.27 g, 28.08 mmol) in dichloromethane (90 mL), under nitrogen atmosphere and in a water bath, was added HMTA (3.94 g, 28.08 mmol) followed by DBU (4.17 mL, 28.08 mmol) and the resulting mixture was stirred for 15 minutes. Then, a solution of (3-{4-Fluoro-2-[3-(4-nonylcarbamoyl-4,5-dihydro-oxazol-2-yl)-7-oxa-bicyclo[2.2.1]hept-2-ylmethyl]-phenyl}-propionic acid methyl ester (3.72 g, 7.02 mmol) in dichloromethane (40 ml) was added and the resulting mixture was stirred at room temperature for 16 h.

After this time, the solution was concentrated under vacuum and the residue was partitioned between ethyl acetate (100 mL) and 1:1 sat. solution of NH₄Cl and NH₃ (100 mL). Then, the organic layer was separated and washed with brine (100 mL), and dried over MgSO₄. Filtration and concentrated in vacuo yield the crude compound as a thick oil.

The residue was purified by column chromatography in silica using Ethyl acetate/iso-hexane 5:1 to isolate the titled compound as a yellow solid (2.2 g, 60%).

¹H-NMR (CDCl₃, 300 MHz) δ 8.07 (s, 1H, ═CH), 7.08 (dd, 1H, J=5.7, 8.6 Hz, ArH), 7.00 (m, 1H, NH), 6.83 (m, 2H, ArH), 4.99 (m, 1H, —CH—O—), 4.37 (m, 1H, —CH—O), 3.66 (s, 3H, CO₂CH₃), 3.39 (m, 3H, —CH— and —CONH—CH₂—), 2.83 (m, 2H, —CH₂—CO₂Me), 2.63-2.48 (m, 3H, —CH₂—CH₂—CO₂Me and —CH—), 2.35 (m, 1H, —CH₂Ar), 2.21 (m, 1H, —CH₂Ar), 1.83 (m, 2H, —CH₂—CH₂—), 1.63-1.25 (m, 4H, —CH₂—CH₂— and —CO—NH—CH₂—CH₂—), 1.25 (m, 12H, —CH₂—CH₂—), 0.91 (m, 3H, —CH₃).

¹⁹F-NMR (CDCl₃, 300 MHz) δ −117)

### Step 8

### 3-{4-Fluoro-2-[3-(4-nonylcarbamoyl-oxazol-2-yl)-7-oxa-bicyclo[2.2.1]hept-2-ylmethyl]-phenyl}-propionic acid

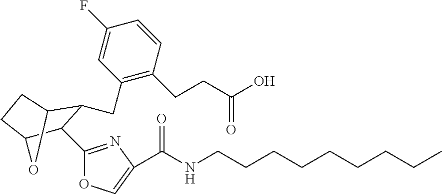 [SMILES: CCCCCCCCCNC(=O)c1coc(C2C3CCC(O3)C2Cc2cc(F)ccc2CCC(=O)O)n1]

To a solution of 3-{4-Fluoro-2-[3-(4-nonylcarbamoyl-oxazol-2-yl)-7-oxa-bicyclo[2.2.1]hept-2-ylmethyl]-phenyl}-propionic acid methyl ester (1.39 g, 2.63 mmol) in tetrahydrofuran (40 ml) was added a solution of Lithium hydroxide (0.441 g, 10.52 mmol) in water (10 mL) and the resulting mixture was stirred at room temperature for 16 h.

After this time, the solution was partitioned between ethyl acetate (100 mL) and 2M HCl solution (50 mL). Then, the organic layer was separated and washed with Brine (50 mL), and dried over MgSO₄. Filtration and concentrated in vacuo yield the titled compound as a light yellow solid. (1.24 g, 92%).

¹H-NMR (CDCl₃, 300 MHz) δ 8.15 (s, 1H, ═CH), 7.17 (m, 1H, NH), 7.10 (dd, 1H, J=5.7, 8.6 Hz, ArH), 6.84 (m, 2H, ArH), 4.99 (m, 1H, —CH—O—), 4.39 (m, 1H, —CH—O), 3.41 (m, 3H, —CH— and —CONH—CH₂—), 2.85 (m, 2H, —CH₂—CO₂Me), 2.62-2.50 (m, 3H, —CH₂—CH₂—CO₂Me and —CH—), 2.36 (m, 1H, —CH₂Ar), 2.20 (m, 1H, —CH₂Ar), 1.84 (m, 2H, —CH₂—CH₂—), 1.63-1.25 (m, 4H, —CH₂—CH₂— and —CO—NH—CH₂—CH₂—), 1.27 (m, 12H, —CH₂—CH₂—), 0.88 (m, 3H, —CH₃).

### Step 9

### 2-{3-[5-Fluoro-2-(3-oxo-3-trifluoromethanesulfonylamino-propyl)-benzyl]-7-oxa-bicyclo[2.2.1]hept-2-yl}-oxazole-4-carboxylic acid nonylamide

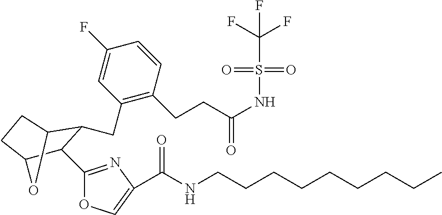 [SMILES: CCCCCCCCCNC(=O)c1coc(C2C3CCC(O3)C2Cc2cc(F)ccc2CCC(=O)NS(=O)(=O)C(F)(F)F)n1]

This compound was prepared from the product of Example 8, Step 8, following general method 1 and using trifluoromethanesulfonamide as reagent. Yield: 60%

¹H-NMR (CDCl₃, 300 MHz) δ 8.19 (s, 1H, ═CH), 7.19 (m, 1H, NH), 7.04 (dd, 1H, J=5.7, 8.6 Hz, ArH), 6.78 (m, 2H, ArH), 4.97 (m, 1H, —CH—O—), 4.35 (m, 1H, —CH—O), 3.45 (m, 1H, —CH—), 3.31 (m, 2H, —CONH—CH₂—), 2.69 (m, 3H, —CH₂—CO₂Me and —CH—), 2.36 (m, 3H, —CH₂—CH₂—CO₂Me and —CH₂Ar), 2.04 (m, 1H, —CH₂Ar), 1.82 (m, 2H, —CH₂—CH₂—), 1.63-1.25 (m, 4H, —CH₂—CH₂— and —CO—NH—CH₂—CH₂—), 1.23 (m, 12H, —CH₂—CH₂—), 0.87 (m, 3H, —CH₃).

¹⁹F-NMR (CDCl₃, 300 MHz) δ −79 and −117

LC-MS: m/z 644 M+H⁺

The compounds of Examples 2 through 8 are tested for FAAH inhibitory activity as follows:

Method 1: Membranes obtained from rat brain are incubated with 2 mM anandamide (N-arachidonoylethanolamine), [¹⁴C]-AEA, 30 min at 37° C. at pH values ranging from 9.00 to 10.00 in presence and absence of tested compounds in a final volume of 500 ml. Incubation is stopped by extraction with CHCl₃/MeOH (1:1) and the aqueous phases containing [¹⁴C]-Ethanolamine produced by [¹⁴C]-AEA hydrolysis is measured.

Method 2: 2 mg/sample of human FAAH recombinant are incubated with 2 mM of [¹⁴C]-AEA for 30 min at 37° C. at pH values ranging from 9.00 to 10.00 in presence and absence of compounds. The final volume of incubation is maintained less than 0.2 ml in order to facilitate enzyme-substrate complex formation. The incubation is stopped by extraction with CHCl₃/MeOH (1:1) and the aqueous phases containing [¹⁴C]-Ethanolamine produced by [¹⁴C]-AEA hydrolysis is measured.

The results of said testing are reported in the Tables, below.

TABLE 1
Example 1

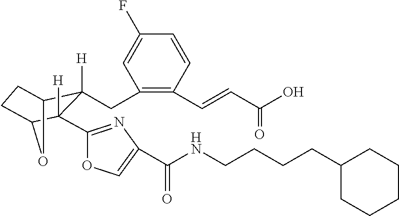 [SMILES: O=C(O)/C=C/c1ccc(F)cc1CC1C2CCC(O2)C1c1nc(C(=O)NCCCCC2CCCCC2)co1]

Example 2

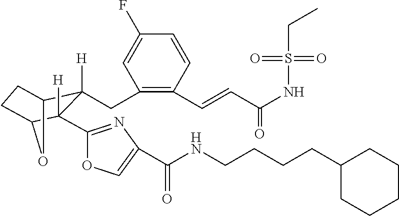 [SMILES: CCS(=O)(=O)NC(=O)/C=C/c1ccc(F)cc1CC1C2CCC(O2)C1c1nc(C(=O)NCCCCC2CCCCC2)co1]

Example 3

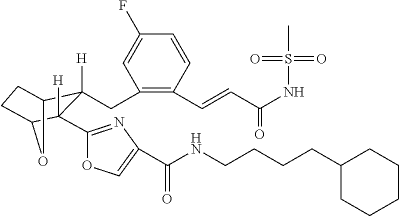 [SMILES: CS(=O)(=O)NC(=O)/C=C/c1ccc(F)cc1CC1C2CCC(O2)C1c1nc(C(=O)NCCCCC2CCCCC2)co1]

Example 4

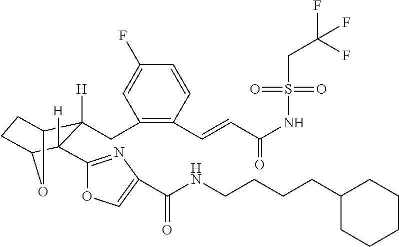 [SMILES: O=C(/C=C/c1ccc(F)cc1CC1C2CCC(O2)C1c1nc(C(=O)NCCCCC2CCCCC2)co1)NS(=O)(=O)CC(F)(F)F]

Example 5

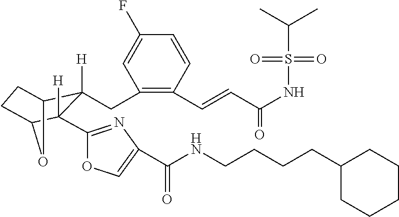 [SMILES: CC(C)S(=O)(=O)NC(=O)/C=C/c1ccc(F)cc1CC1C2CCC(O2)C1c1nc(C(=O)NCCCCC2CCCCC2)co1]

Example 6

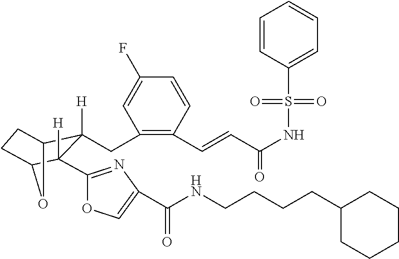 [SMILES: O=C(/C=C/c1ccc(F)cc1CC1C2CCC(O2)C1c1nc(C(=O)NCCCCC2CCCCC2)co1)NS(=O)(=O)c1ccccc1]

Example 7

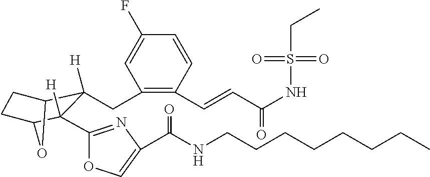 [SMILES: CCCCCCCCNC(=O)c1coc(C2C3CCC(O3)C2Cc2cc(F)ccc2/C=C/C(=O)NS(=O)(=O)CC)n1]

Example 8

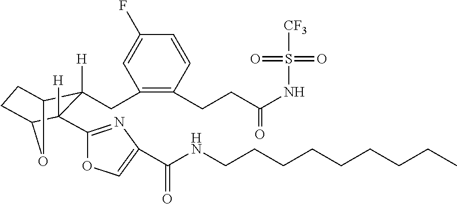 [SMILES: CCCCCCCCCNC(=O)c1coc(C2C3CCC(O3)C2Cc2cc(F)ccc2CCC(=O)NS(=O)(=O)C(F)(F)F)n1]

| Example | | | | | | | | | |
|---|---|---|---|---|---|---|---|---|---|
| number | FAAH | FP | DP | EP1 | EP2 | EP3 | EP4 | IP | TP |
| Example 1 | 2700 | 60 | 70 | 380 | NA | 5500 | 60 | NA | <1 |
| Example 2 | 400 | 100 | 50 | 190 | NA | 1600 | 140 | 1100 | <1 |
| Example 3 | 600 | 90 | 30 | 65 | NA | 790 | 100 | NA | <1 |
| Example 4 | 700 | 130 | 80 | 50 | 5300 | 2500 | 80 | NA | <1 |
| Example 5 | 100 | 220 | 50 | 30 | NA | 1500 | 300 | 750 | <1 |
| Example 6 | 80 | 100 | 60 | 60 | NA | 290 | 380 | 1500 | <1 |
| Example 7 | 200 | 370 | 45 | 80 | NA | 1900 | 200 | 3500 | <1 |
| Example 8 | 300 | 50 | 10 | 40 | 4000 | 700 | 30 | 3300 | <1 |

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. In particular, while the present invention has been illustrated by the treatment of pain, the method of using the above compounds to treat any of the diseases and/or conditions of humans that are mediated by FAAH and/or the above described PG receptors, especially conditions that benefit from blocking and antagonizing both the FAAH inhibiting activity and the activity at one or more PG receptors, e.g. the DP₁, FP, EP₁, EP₃, TP, and/or EP₄ receptors, are within the scope of this invention. Also, although the present invention is illustrated by the specific compounds of Examples 2 through 8, the following compounds may be used in the method of the present invention:

3-[4-[[(Hydrocarbyl or substituted hydrocarbyl)amino]carbonyl]-2-oxazolyl]-7-oxabicyclo[2.2.1]hept-2-yl]methyl]-substituted-phenyl]-N-(ethylsulfonyl)acrylic amide

3-[4-[[(Alkyl)amino]carbonyl]-2-oxazolyl]-7-oxabicyclo[2.2.1]hept-2-yl]methyl]-4-substituted-phenyl]-N-(ethylsulfonyl)acrylic amide

3-[4-[[(Alkyl)amino]carbonyl]-2-oxazolyl]-7-oxabicyclo[2.2.1]hept-2-yl]methyl]-4-halo-phenyl]-N-(ethylsulfonyl)acrylic amide.

It is intended that all such modifications will fall within the scope of the method of treatment claims and the compound claims hereof.

What is claimed is:

1. A method of treating a patient suffering from pain by administering a fatty acid amide inhibiting amount of a compound represented by the formula: 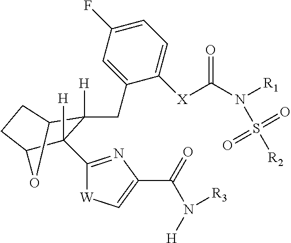
wherein R₁ is H;
R₂ is a radical selected from the group consisting of H, hydrocarbyl and substituted hydrocarbyl;
R₃ is a radical selected from the group consisting of H, hydrocarbyl and substituted hydrocarbyl;
X is CHCH, (CH₂)ₙ or O(CH₂)ₙ, wherein n is 0 or an integer of from 1 to 4; and
W is O, S, or NR⁶, wherein R⁶ is selected from the group consisting of H and alkyl.

2. The method of claim 1 wherein W is O.

3. The method of claim 1 wherein R₂ is a radical selected from the group consisting of H, alkyl, haloalkyl and aryl.

4. The method of claim 2 wherein R₂ is selected from the group consisting of ethyl, methyl, 2-methylethyl, phenyl, trifluoromethyl and 2, 2, 2 trifluoroethyl.

5. The method of claim 1 wherein R₃ is selected from the group consisting of H, alkyl, alkenyl and aryl.

6. The method of claim 4 wherein R₃ is selected from the group consisting of n-alkyl and cycloalkyl-n-alkyl

7. The method of claim 5 wherein, R₃ is (CH₂)ₙCH₂R₅, wherein n is an integer of from 4 to 9 and R₅ is H or cycloalkyl.

8. The method of claim 6 wherein R₃ is selected from the group consisting of cyclohexyl-n-alkyl radicals.

9. The method of claim 7 wherein R₃ is cyclohexyl-n-butyl.

10. The method of claim 1 wherein X is ethyl or ethenyl.

11. The method of claim 1 wherein said compound is selected from the group consisting of
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(ethylsulfonyl)acrylic amide,
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(methylsulfonyl)acrylic amide,
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2,2,2-trifluoroethanesulfonyl)acrylic amide,
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2-methylethanesulfonyl)acrylic amide,
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(benzenesulfonyl)acrylic amide,
(E)-3R-[2R-[[3-[4-[[(octylamino]carbonyl]-2-oxazolyl]-7-oxabicyclo[2.2.1]hept-2-yl]methyl]-4-fluoro-phenyl]-N-(ethylsulfonyl)acrylic amide, and
2-{3-[5-Fluoro-2-(3-oxo-3-trifluoromethanesulfonylamino-propyl)-benzyl]-7-oxa-bicyclo[2.2.1]hept-2-yl}-oxazole-4-carboxylic acid nonylamide.

12. A method of treating a patient having a condition mediated by FAAH which comprises administering a fatty acid amide inhibiting amount of a compound represented by the formula: 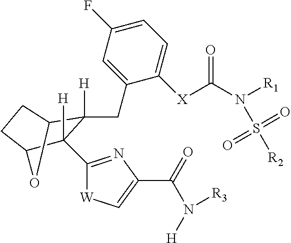
wherein R1 is H;
R2 is a radical selected from the group consisting of H, hydrocarbyl and substituted hydrocarbyl;
R3 is a radical selected from the group consisting of H, hydrocarbyl and substituted hydrocarbyl;
X is CHCH, (CH2)n or O(CH2) n, wherein n is 0 or an integer of from 1 to 4; and
W is O, S, or NR6, wherein R6 is selected from the group consisting of H and alkyl.

13. A method of treating a patient having a condition mediated by FAAH and at least one PG receptor which comprises administering a fatty acid amide inhibiting amount of a compound represented by the formula 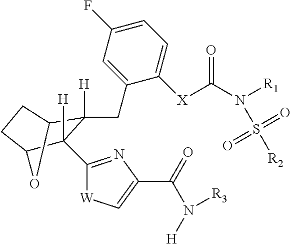
wherein R₁ is H;
R₂ is a radical selected from the group consisting of H, hydrocarbyl and substituted hydrocarbyl;
R₃ is a radical selected from the group consisting of H, hydrocarbyl and substituted hydrocarbyl;
X is CHCH, (CH₂)ₙ or O(CH₂)ₙ, wherein n is 0 or an integer of from 1 to 4; and
W is O, S, or NR⁶, wherein R⁶ is selected from the group consisting of H and alkyl.

14. The method of claim 13 wherein said condition is a pain-related condition.

15. A method of treating a patient suffering from pain by administering a fatty acid amide inhibiting amount of a compound, that is a 3-[4-[[(hydrocarbyl or substituted hydrocarbyl)amino]carbonyl]-2-oxazolyl]-7-oxabicyclo[2.2.1]hept-2-yl]methyl]-substitutedphenyl]-N-(ethylsulfonyl)acrylic amide.

16. The method of claim 15 wherein said compound is a 3-[4-[[(alkyl)amino]carbonyl]-2-oxazolyl]-7-oxabicyclo[2.2.1]hept-2-yl]methyl]-4-substituted-phenyl]-N-(ethylsulfonyl)acrylic amide.

17. The method of claim 15 wherein said compound is a 3-[4-[[(alkyl)amino]carbonyl]-2-oxazolyl]-7-oxabicyclo[2.2.1]hept-2-yl]methyl]-4-halo-phenyl]-N-(ethylsulfonyl)acrylic amide.

18. A compound represented by the formula: 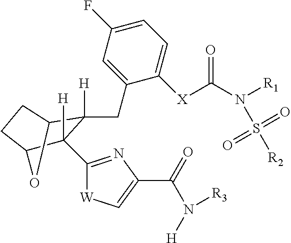
wherein R₁ is H;
R₂ is a radical selected from the group consisting of H, hydrocarbyl and substituted hydrocarbyl;
R₃ is a radical selected from the group consisting of H, hydrocarbyl and substituted hydrocarbyl;
X is CHCH, (CH₂)ₙ or O(CH₂)ₙ, wherein n is 0 or an integer of from 1 to 4; and
W is O, S, or NR⁶, wherein R⁶ is selected from the group consisting of H and alkyl.

19. The compound of claim 18 wherein W is O.

20. The compound of claim 19 wherein R₂ is a radical selected from the group consisting of H, alkyl, haloalkyl and aryl.

21. The compound of claim 19 wherein R₂ is selected from the group consisting of ethyl, methyl, 2-methylethyl, phenyl, trifluoromethyl and 2, 2, 2 trifluoroethyl.

22. The compound of claim 18 wherein R₃ is selected from the group consisting of H, alkyl, alkenyl and aryl.

23. The compound of claim 21 wherein R₃ is selected from the group consisting of n-alkyl and cycloalkyl-n-alkyl

24. The compound of claim 22 wherein R₃ is (CH₂)ₙCH₂R₅, wherein n is an integer of from 4 to 9 and R₅ is H or cycloalkyl.

25. The compound of claim 23 wherein R₃ is selected from the group consisting of cyclohexyl-n-alkyl radicals.

26. The compound of claim 18 wherein R₃ is cyclohexyl-n-butyl.

27. The compound of claim 18 wherein X is ethyl or ethenyl.

28. The compound of claim 18 wherein said compound is selected from the group consisting of:
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(ethylsulfonyl)acrylic amide;
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(methylsulfonyl)acrylic amide;
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2,2,2-trifluoroethanesulfonyl)acrylic amide;
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(2-methylethanesulfonyl)acrylic amide;
(E)-3R-[2R-[[3-[4-[[(4-cyclohexylbutyl)amino]carbonyl]-2-oxazolyl]-7-oxabicyclo[2.2.1]hept-2-yl]methyl]-4-fluoro-phenyl]-N-(benzenesulfonyl)acrylic amide;
(E)-3R-[2R-[[3-[4-[[(octylamino]carbonyl]-2-oxazolyl]-7-oxabicyclo[2.2.1]hept-2-yl]methyl]-4-fluoro-phenyl]-N-(ethylsulfonyl)acrylic amide; and,
2-{3-[5-Fluoro-2-(3-oxo-3-trifluoromethanesulfonylamino-propyl)-benzyl]-7-oxa-bicyclo[2.2.1]hept-2-yl}-oxazole-4-carboxylic acid nonylamide.
